(19) United States
(12) Patent Application Publication
(10) Pub. No.: US 2023/0215586 A1
(43) Pub. Date: Jul. 6, 2023

(54) Monitoring, Tracing, Early-Warning And Control System And Method For Virus Spreading In Building

(71) Applicant: TSINGHUA UNIVERSITY, Beijing (CN)

(72) Inventors: Borong Lin, Beijing (CN); Hao Zhou, Beijing (CN); Zhongchen Zhang, Beijing (CN); Hongli Sun, Beijing (CN); Li Liu, Beijing (CN); Juan Yu, Beijing (CN)

(21) Appl. No.: 17/927,392

(22) PCT Filed: Mar. 18, 2021
(86) PCT No.: PCT/CN2021/081475

§ 371 Date: Feb. 15, 2023

(30) Foreign Application Priority Data
May 27, 2020 (CN) 202010460230.1

(51) Int. Cl. G16H 50/80

(52) U.S. Cl. CPC G16H 50/80

(57) ABSTRACT

The present disclosure provides a monitoring, tracing, early-warning and control system and method for virus spreading in a building. Indoor and outdoor environmental parameters of the building can be monitored in real time. Human physiological indexes are obtained in real time to determine a human health state, and moving trajectories of people and droplet spreading trajectories are obtained. Therefore, suspected infected persons and their close contacts are timely and accurately identified, and countermeasures are taken for the suspected infected persons and the close contacts. In addition, a spreading risk in a current indoor environment can be assessed, the indoor environment is ventilated and purified according to a spreading risk assessment result, and local areas of the suspected infected persons and the close contacts are effectively ventilated and purified, so as to reduce the spreading risk.

### CROSS REFERENCE TO RELATED APPLICATION

The present application claims priority to the Chinese Patent Application No. 202010460230.1, filed with the China National Intellectual Property Administration (CNIPA) on May 27, 2020, and entitled “MONITORING, TRACING, EARLY-WARNING AND CONTROL SYSTEM AND METHOD FOR VIRUS SPREADING IN BUILDING”, which is incorporated herein by reference in its entirety.

### TECHNICAL FIELD

The present disclosure relates to the field of monitoring, tracing, early-warning and control of virus spreading, and in particular, to a monitoring, tracing, early-warning and control system and method for virus spreading in a building.

### BACKGROUND

At present, building control usually takes the environmental threshold set by users as the control target, and considers the room as a whole according to the lumped parameter method. With the emergence of the coronavirus disease 2019 (COVID-19), this control method is difficult to meet the epidemic prevention control target since it ignores local environmental parameters. Overall, there are the following problems.

First, the target parameters set by the user for control are single, and the control target is unreasonable. Physical quantities such as temperature and carbon dioxide concentration are often used as control parameters of heating, ventilation and air-conditioning (HVAC) equipment. Although these parameters are easily available and quickly responded by the equipment, they are difficult to reflect the exposure risk in the environment of virus spreading. In addition, it is difficult for users to select the optimal control target, so that the indoor environmental control is difficult to meet the requirements of epidemic prevention and control.

Second, indoor environmental monitoring means are single. The current control considers the entire room as a whole according to the lumped parameter method, ignoring the field distribution and the spreading path of pollutants in the space. However, in the environment of virus spreading, the source and spreading path have an important impact on the exposure risk of the virus. The traditional monitoring means measures indoor environmental parameters by one measuring point in each control unit. In this case, it is difficult to determine the moving trajectory of the source and the spreading path of pollutants, so it is difficult to carry out targeted control.

Third, the monitoring and control ignore local areas and may underestimate exposure risks. The monitoring and control based on the lumped parameter method has another disadvantage that the parameter of measuring point position is used as the parameter of the whole control unit. This may underestimate the concentration of pollutants in the space near the source and the inaccessible space of ventilation and purification, thus underestimating the exposure risk and making it difficult to carry out targeted control.

In the current situation of COVID-19, buildings are confined spaces with high density of people, so the spreading and prevention of COVID-19 in buildings is an important work in the future. Similar aerosol-spread viruses have similar needs. Therefore, the present disclosure mainly studies the virus spread by aerosols (hereinafter referred to as the virus), and provides a new technology based on a combination of environmental monitoring in buildings and identification of human health characteristics, which can realize the monitoring, tracing, and early-warning control of virus spreading in buildings.

### SUMMARY

In order to overcome the above shortcomings of the prior art, the present disclosure provides a monitoring, tracing, early-warning and control system and method for virus spreading in a building, which overcomes the following defects in the prior art and adopts a new technology combining environmental monitoring in buildings and identification of human health characteristics to realize the monitoring, tracing, and early-warning control of virus spreading in the building. The defects in the prior art are as follows: first, when an environmental threshold set by users is taken as a control target and an indoor environment is considered by the lumped parameter method, target parameters are difficult to reflect a spreading risk of the virus, and indoor environmental control is difficult to meet an epidemic prevention control target. Second, indoor environmental monitoring means are single, and it is difficult to determine a moving trajectory of a virus source and a spreading path of pollutants. Third, a parameter of a whole control unit is obtained by performing monitoring and control using the lumped parameter method, ignoring a local environment, so as to underestimate an exposure risk of the virus. The present disclosure mainly aims at monitoring, tracing, early-warning and control of spreading of a virus spread by aerosols in buildings.

A technical solution adopted by the present disclosure to solve the technical problems is: the present disclosure provides a monitoring, tracing, early-warning and control system for virus spreading in a building, including an environmental monitoring system, a virus tracing system, an environmental diagnosis and early warning system, and a ventilation and purification linkage control system. The environmental monitoring system includes a static environmental monitoring system and a dynamic monitoring system.

The static environmental monitoring system is configured to monitor indoor and outdoor environmental parameters of the building. The environmental parameters include temperature and humidity and a pollutant concentration.

The dynamic monitoring system is configured to monitor human physiological indexes, dynamic indexes of people, and a distribution of an indoor object surface temperature field. The dynamic indexes of people include a droplet spreading trajectory and a moving trajectory of people.

The virus tracing system is configured to identify and determine suspected infected persons according to the obtained human physiological indexes to obtain an identification and determination result, analyze a droplet spreading trajectory of the suspected infected person and the moving trajectory of people when the identification and determination result is the suspected infected person to obtain an analysis result, determine close contacts according to the analysis result, and take countermeasures for the close contacts and the suspected infected person.

The environmental diagnosis and early warning system is configured to establish a current indoor environmental field, correct the current indoor environmental field according to the indoor and outdoor environmental parameters of the building to obtain an indoor environmental field subjected to current correction, perform spreading risk assessment in combination with an indoor environmental field subjected to historical correction and the droplet spreading trajectory and the moving trajectory of the suspected infected person to obtain a spreading risk assessment result, and perform high risk early warning based on the spreading risk assessment result. The indoor environmental field subjected to the historical correction is a corrected indoor environmental field before establishment of the current indoor environmental field.

The ventilation and purification linkage control system is configured for ventilation and purification linkage control according to the identification and determination result, the analysis result, and the spreading risk assessment result.

Optionally, the static environmental monitoring system includes a temperature sensor, a humidity sensor, and a pollutant concentration measurement sensor. The dynamic monitoring system includes an infrared camera.

Optionally, the environmental diagnosis and early warning system includes an indoor environmental field establishment module, an indoor environmental field correction module, and a spreading risk assessment module.

The indoor environmental field establishment module is configured to simulate a distribution of the indoor environmental parameters using building environment simulation software, so as to establish the current indoor environmental field.

The indoor environmental field correction module is configured to correct the established current indoor environmental field based on the indoor environmental parameters obtained by the static environmental monitoring system in combination with the distribution of the object surface temperature field, so as to obtain the indoor environmental field subjected to the current correction.

The spreading risk assessment module is configured to calculate an infection probability of indoor virus spreading according to the indoor environmental field subjected to the current correction, the indoor environmental field subjected to the historical correction, and the droplet spreading trajectory and the moving trajectory of the suspected infected person, so as to assess a spreading risk. A greater infection probability indicates a greater spreading risk.

Optionally, the indoor environmental field establishment module is configured to simulate a distribution of the indoor environmental parameters using building environment simulation software, so as to establish the current indoor environmental field; specifically, the indoor environmental field establishment module is configured to:

establish a building physical model, and simulate the distribution of the current indoor environmental parameters using the building environment simulation software in combination with a real-time operating state of building environment control equipment and real-time positioning information of people, so as to establish the current indoor environmental field, where the current indoor environmental field includes a temperature and humidity field, a pollutant concentration field, and an air distribution.

Optionally, the ventilation and purification linkage control system includes an overall control system and a local control system.

The overall control system is configured for overall optimization control based on the spreading risk assessment result to realize overall ventilation and purification of the building.

The local control system is configured for local ventilation and purification of an area where a concentration of a pollutant in the indoor environmental field subjected to the current correction is higher than a set threshold, and local purification and dilution ventilation of an area where the suspected infected person is located and an area where the close contacts are located to realize local purification treatment of a source.

Optionally, the monitoring, tracing, early-warning and control system for virus spreading in a building further includes a ventilation and purification effect assessment system configured to assess a ventilation and purification effect of the ventilation and purification linkage control system; specifically, the ventilation and purification effect assessment system is configured to:

establish an environmental field-time function through the indoor environmental field subjected to the current correction and the indoor environmental field subjected to the historical correction, calculate an attenuation rate of the pollutant concentration, and assess the ventilation and purification effect according to the attenuation rate of the pollutant concentration and a current pollutant concentration.

The present disclosure further provides a monitoring, tracing, early-warning and control method for virus spreading in a building, including the following steps:

step I: obtaining indoor and outdoor environmental parameters of the building, where the environmental parameters include temperature and humidity and a pollutant concentration; and

obtaining human physiological indexes, dynamic indexes of people, and a distribution of an indoor object surface temperature field, where the dynamic indexes of people include a droplet spreading trajectory and a moving trajectory of people;

step II: identifying and determining suspected infected persons according to the obtained human physiological indexes to obtain an identification and determination result, analyzing a droplet spreading trajectory of the suspected infected person and the moving trajectory of people when the identification and determination result is the suspected infected person to obtain an analysis result, determining close contacts according to the analysis result, and taking countermeasures for the close contacts and the suspected infected person;

step III: establishing a current indoor environmental field, correcting the current indoor environmental field according to the indoor and outdoor environmental parameters of the building to obtain an indoor environmental field subjected to current correction, performing spreading risk assessment in combination with an indoor environmental field subjected to historical correction and the droplet spreading trajectory and the moving trajectory of the suspected infected person to obtain a spreading risk assessment result, and performing high risk early warning based on the spreading risk assessment result, where the indoor environmental field subjected to the historical correction is a corrected indoor environmental field before establishment of the current indoor environmental field;

step IV: performing ventilation and purification linkage control according to the identification and determination result, the analysis result, and the spreading risk assessment result; and

step V: assessing a ventilation and purification linkage control effect.

Optionally, a process of correcting the current indoor environmental field according to the indoor and outdoor environmental parameters of the building and performing spreading risk assessment in combination with an indoor environmental field subjected to historical correction and the droplet spreading trajectory and the moving trajectory of the suspected infected person to obtain a spreading risk assessment result specifically includes:

establishing a building physical model, and simulating the distribution of the current indoor environmental parameters using building environment simulation software in combination with a real-time operating state of building environment control equipment and real-time positioning information of people, so as to establish the current indoor environmental field, where the current indoor environmental field includes a temperature and humidity field, a pollutant concentration field, and an air distribution;

correcting the established current indoor environmental field based on the obtained indoor environmental parameters in combination with the distribution of the indoor object surface temperature field, so as to obtain the indoor environmental field subjected to the current correction; and

calculating an infection probability of indoor virus spreading according to the indoor environmental field subjected to the current correction, the indoor environmental field subjected to the historical correction, and the droplet spreading trajectory and the moving trajectory of the suspected infected person, so as to assess a spreading risk, where a greater infection probability indicates a greater spreading risk.

Optionally, a process of performing ventilation and purification linkage control according to the identification and determination result, the analysis result, and the spreading risk assessment result specifically includes:

overall control: performing overall optimization control based on the spreading risk assessment result to realize overall ventilation and purification of the building; and

local control: performing local ventilation and purification on an area where a concentration of a pollutant in the indoor environmental field subjected to the current correction is higher than a set threshold and local purification and dilution ventilation on an area where the suspected infected person is located and an area where the close contacts are located to realize local purification treatment of a source.

Optionally, a process of assessing a ventilation and purification linkage control effect specifically includes:

establishing an environmental field-time function through the indoor environmental field subjected to the current correction and the indoor environmental field subjected to the historical correction, calculating an attenuation rate of the pollutant concentration, and assessing the ventilation and purification effect according to the attenuation rate of the pollutant concentration and a current pollutant concentration.

The present disclosure has the following advantages compared with the prior art: the present disclosure provides a monitoring, tracing, early-warning and control system and method for virus spreading in a building. Indoor and outdoor environmental parameters of the building can be monitored in real time. Human physiological indexes are obtained in real time to determine a human health state, and moving trajectories of people and droplet spreading trajectories are obtained. Therefore, suspected infected persons and their close contacts are timely and accurately identified, and countermeasures are timely taken for the suspected infected persons and the close contacts. In addition, a spreading risk in a current indoor environment can be assessed. Overall ventilation and purification is performed on the indoor environment according to a spreading risk assessment result. Local ventilation and purification is performed on an area where a concentration of a pollutant in the indoor environmental field subjected to the current correction is higher than a set threshold. Local areas of the suspected infected persons and the close contacts are effectively ventilated and purified, so as to reduce the spreading risk. Besides, the ventilation and purification effect can be assessed. In summary, the method combines indoor environmental parameters of the building, human health characteristics and virus spreading risk assessment. Thus, an indoor environment state of the building can be accurately determined, and the suspected infected persons and the close contacts can be accurately identified in real time. Based on an indoor environmental health effect and the status of suspected infected persons, indoor overall ventilation and purification and local purification treatment are performed.

### BRIEF DESCRIPTION OF THE DRAWINGS

The present disclosure will be further described below with reference to the accompanying drawings.

FIG. 1 is a system structure diagram of a monitoring, tracing, early-warning and control system for virus spreading in a building provided by Embodiment 1 of the present disclosure;

FIG. 2 is a system schematic diagram of the monitoring, tracing, early-warning and control system for virus spreading in a building provided by Embodiment 1 of the present disclosure; and

FIG. 3 is a method flow chart of a monitoring, tracing, early-warning and control method for virus spreading in a building provided by Embodiment 2 of the present disclosure.

### DETAILED DESCRIPTION OF THE EMBODIMENTS

An objective of the present disclosure is to provide a monitoring, tracing, early-warning and control system and method for virus spreading in a building, which overcomes the following defects in the prior art and adopts a new technology combining environmental monitoring in buildings and identification of human health characteristics to realize the monitoring, tracing, and early-warning control of virus spreading in the building. The defects in the prior art are as follows: first, when an environmental threshold set by users is taken as a control target and an indoor environment is considered by the lumped parameter method, target parameters are difficult to reflect a spreading risk of the virus, and indoor environmental control is difficult to meet an epidemic prevention control target. Second, indoor environmental monitoring means are single, and it is difficult to determine a moving trajectory of a virus source and a spreading path of pollutants. Third, a parameter of a whole control unit is obtained by performing monitoring and control using the lumped parameter method, ignoring a local environment, so as to underestimate an exposure risk of the virus.

The present disclosure is further described below with reference to the accompanying drawings and embodiments.

### Embodiment 1

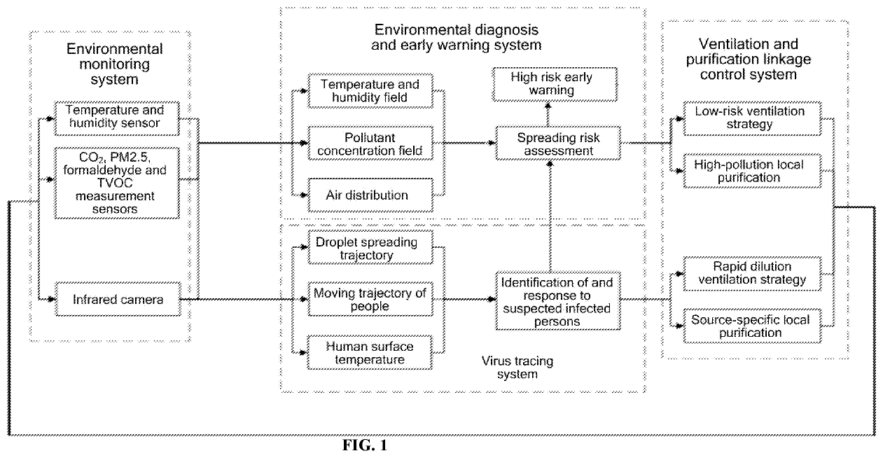

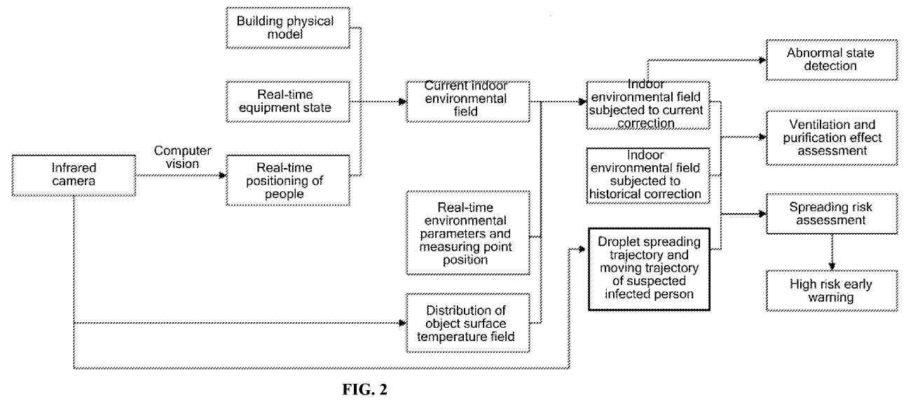

As shown in FIG. 1 and FIG. 2, a monitoring, tracing, early-warning and control system for virus spreading in a building includes an environmental monitoring system, a virus tracing system, an environmental diagnosis and early warning system, and a ventilation and purification linkage control system. The environmental monitoring system includes a static environmental monitoring system and a dynamic monitoring system.

The static environmental monitoring system is configured to monitor indoor and outdoor environmental parameters of the building.

The environmental parameters include, but are not limited to, temperature and humidity and a pollutant concentration.

The static environmental monitoring system includes, but is not limited to, a temperature sensor, a humidity sensor, and a pollutant concentration measurement sensor.

The pollutant concentration measurement sensor includes, but is not limited to, CO₂, PM2.5, formaldehyde and total volatile organic compound (TVOC) measurement sensors. Meanwhile, the pollutant concentration specifically includes, but is not limited to, a CO₂ concentration, a PM2.5 concentration, a formaldehyde concentration, and a TVOC concentration.

In addition, the static environmental monitoring system needs to be installed inside and outside the building at the same time, which can monitor various indoor and outdoor real-time environmental parameters, such that the monitored environmental parameters are more comprehensive.

The dynamic monitoring system is configured to monitor human physiological indexes, dynamic indexes of people, and a distribution of an indoor object surface temperature field. The dynamic indexes of people include a droplet spreading trajectory and a moving trajectory of people.

For the selection of human physiological indexes, it can be either the human surface temperature or any human physiological characteristics, without any specific restrictions. The dynamic indexes of people also include, but are not limited to, a droplet spreading trajectory and a moving trajectory of people.

The dynamic monitoring system includes an infrared camera. In order to flexibly monitor the human physiological indexes, the dynamic indexes of people and the distribution of the indoor object surface temperature field, the dynamic monitoring system can also use other high-precision cameras. As long as the monitoring requirements can be met, any equipment with similar functions is within the protection scope of the present disclosure.

The virus tracing system is configured to identify and determine suspected infected persons according to the obtained human physiological indexes to obtain an identification and determination result, analyze a droplet spreading trajectory of the suspected infected person and the moving trajectory of people when the identification and determination result is the suspected infected person to obtain an analysis result, determine close contacts according to the analysis result, and take countermeasures for the close contacts and the suspected infected person.

The present embodiment is illustrated by using the basic human temperature as the human physiological index and the infrared camera as the dynamic monitoring system. When the surface temperature of a person identified by the infrared camera is higher than a certain threshold (such as 37.3° C.), the person is identified as a suspected infected person. Meanwhile, the moving trajectory of the suspected infected person recorded by the infrared camera shall be analyzed to quickly determine the specific location of the suspected infected person and further analyze the droplet spreading trajectory of the suspected infected person. People within a certain range of the above droplet spreading trajectory and moving trajectory can be identified as close contacts. In this case, it is necessary to take countermeasures for the suspected infected person and the close contacts timely, such as quarantine observation or hospitalization. By using the dynamic monitoring system to monitor the physiological indexes, moving trajectory and droplet spreading trajectory of people in real time, the suspected infected persons and their close contacts can be identified in a timely, reliable and accurate manner, and corresponding measures can be taken to effectively avoid effectively prevent the virus from continuing to spread among indoor populations.

It should be noted that unlike the infrared imager, the infrared camera can continuously shoot infrared images. Through computer vision technology, the relative position relationship and body similarity between two adjacent frames of people identification can be determined. The problem of separating people positions after overlapping has also been realized technically. Therefore, the video obtained by the infrared camera can determine the moving trajectories of different people. In addition, since the target is a suspected infected person, the accuracy of the obtained moving trajectory identification results will be further improved without considering the trajectory of other people.

The identification of and response to suspected infected persons include two parts: identifying suspected infected persons due to high body temperature and identifying their moving trajectories in the building so that managers can quickly locate and take countermeasures against them. The results of moving trajectory identification have no direct impact on the identification and determination of infected persons, but are reflected in the countermeasures taken for suspected infected persons.

The environmental diagnosis and early warning system is configured to establish a current indoor environmental field, correct the current indoor environmental field according to the indoor and outdoor environmental parameters of the building to obtain an indoor environmental field subjected to current correction, perform spreading risk assessment in combination with an indoor environmental field subjected to historical correction and the droplet spreading trajectory and the moving trajectory of the suspected infected person to obtain a spreading risk assessment result, and perform high risk early warning based on the spreading risk assessment result. The indoor environmental field subjected to the historical correction is a corrected indoor environmental field before establishment of the current indoor environmental field.

The environmental diagnosis and early warning system includes an indoor environmental field establishment module, an indoor environmental field correction module, and a spreading risk assessment module.

The indoor environmental field establishment module is configured to simulate a distribution of the indoor environmental parameters using building environment simulation software, so as to establish the current indoor environmental field.

This is implemented specifically in the following steps: establishing a building physical model, and simulating the distribution of the current indoor environmental parameters using the building environment simulation software in combination with a real-time operating state of building environment control equipment and real-time positioning information of people, so as to establish the current indoor environmental field. The current indoor environmental field includes a temperature and humidity field, a pollutant concentration field, and an air distribution.

The air distribution mainly refers to the air flow field distribution in a room, which is obtained mainly by cloud simulation, that is, obtained through the input of information such as room model, air outlet state parameters, air speed and air volume by computational fluid dynamics. The air outlet state parameters include a temperature and relative humidity. Air outlet parameters of a purifier also include parameters such as a PM2.5 concentration. In these state parameters, the temperature has a significant effect on the air distribution. To put it simply, the air distribution is obtained mainly by taking the monitored temperature and air volume and air speed as the boundary conditions, and using computational fluid dynamics under the framework of the room model for cloud simulation.

The indoor environmental field correction module is configured to correct the established current indoor environmental field based on the indoor environmental parameters obtained by the static environmental monitoring system in combination with the distribution of the object surface temperature field, so as to obtain the indoor environmental field subjected to the current correction.

Considering that the current indoor environmental field is obtained through environmental simulation software, the relative relationship between its values has a certain reliability, but there may be deviation between the absolute value and the real result. Therefore, it needs to be corrected. In addition to the correction basis given above, the measuring point position can also be considered in the correction. In addition, although the actual static environmental monitoring system can only place one or a limited number of devices in the same space due to the limitation of the number of sensors, it cannot accurately reflect the environmental field condition, but because of its accurate and reliable test results, it is used to correct the simulation results of environmental field.

Different correction processes can be adopted for different parameters. A relatively simple method is listed here. According to the test results of several sensors in the space, it is considered to be the true value of the corresponding point of the environmental field, and the correction value of each point in the space is obtained by weighted correction according to the proportion of distance. This method is the simplest correction method, only to clearly explain the correction process of the present disclosure. The correction process involved in the present disclosure can also be other correction methods, and the above correction methods have no limiting effect on the present disclosure.

The spreading risk assessment module is configured to calculate an infection probability of indoor virus spreading according to the indoor environmental field subjected to the current correction, the indoor environmental field subjected to the historical correction, and the droplet spreading trajectory and the moving trajectory of the suspected infected person, so as to assess a spreading risk. A greater infection probability indicates a greater spreading risk.

In the present embodiment, the risk assessment can be performed by calculating the infection probability of indoor virus spreading through models including, but not limited to, the Wells-Riley model and the pollutant spreading risk model based on dilution rate. A greater infection probability indicates a greater spreading risk.

The ventilation and purification linkage control system is configured for ventilation and purification linkage control according to the identification and determination result, the analysis result, and the spreading risk assessment result.

The ventilation and purification linkage control system includes an overall control system and a local control system.

The overall control system is configured for overall optimization control based on the spreading risk assessment result to realize overall ventilation and purification of the building.

When the overall spreading risk is low, the low-risk ventilation strategy is adopted to perform overall ventilation and purification on the building.

The local control system is configured for local ventilation and purification of an area where a concentration of a pollutant in the indoor environmental field subjected to the current correction is higher than a set threshold, and local purification and dilution ventilation of an area where the suspected infected person is located and an area where the close contacts are located to realize local purification treatment of a source.

In the indoor environmental field after the current correction, the concentration of pollutants in local areas may be higher than the set threshold, such that local areas are in a high pollution state, so high pollution local purification is required. In addition, since the suspected infected person and close contacts may be the source of virus spreading, it is also necessary to perform source-specific local purification on their locations and adopt the rapid dilution ventilation strategy.

In order to timely understand the effect of indoor ventilation and purification, the system can further designs a ventilation and purification effect assessment system configured to assess a ventilation and purification effect of the ventilation and purification linkage control system specifically in the following steps.

An environmental field-time function is established (for example, a time series of parameters such as CO₂ and PM2.5 is established) through the indoor environmental field subjected to the current correction and the indoor environmental field subjected to the historical correction, an attenuation rate of the pollutant concentration is calculated, and the ventilation and purification effect is assessed according to the attenuation rate of the pollutant concentration and a current pollutant concentration.

The ventilation and purification solution is adjusted for local areas with a low attenuation rate and a high current pollutant concentration, so as to realize rapid and targeted ventilation and purification. In addition, the infection probability of current indoor virus spreading can be calculated. A greater infection probability indicates a poorer ventilation and purification effect. Therefore, it is necessary to perform high risk early warning and readjust ventilation and purification measures, such as indoor overall ventilation and purification and targeted and rapid ventilation and purification of high-pollution areas. In addition, the ventilation and purification effect can be assessed by the frequency of air exchange. It should be noted that the ventilation and purification assessment parameters mentioned above have no limiting effect on the present disclosure.

In addition, it should be added that it can also detect the abnormal state of the indoor environmental field after the current correction. Therefore, the environmental diagnosis and early warning system can also include an abnormal state detection module, and the abnormal state detection mainly includes outlier detection and high concentration detection. The outlier detection is mainly aimed at the situation where local space environmental parameters are too different from other spaces due to unreasonable air distribution or ventilation purification strategy. The high concentration detection is aimed at the deviation of some parameters of local space or the whole space from the control domain, which will cause harm to human comfort and health.

In the case of outlier detection, if the local space is located in the active area of people and the environmental parameter outlier is not conducive to the health of people, an alarm is given and local ventilation and purification measures are added. If the local space is not located in the active area of people and the environmental parameter outlier is not conducive to the health of the people, only an alarm is given and the management personnel increases the local ventilation and purification measures as required. If the local space is located in the comfort zone of people and the environmental parameter outlier is conducive to the health of people, ventilation and purification treatment are not performed.

In the case of high concentration detection, if the overall spatial parameters deviate from the control domain, the overall ventilation and purification air volume is increased or the air supply parameters are adjusted to ensure a healthy and comfortable overall space. If the local spatial parameters deviate from the control domain, the local ventilation and purification air volume is adjusted or the air supply parameters are adjusted to ensure a healthy and comfortable local space. For the space that cannot be adjusted locally, the health and comfort near the real-time location of people is prioritized.

In the present embodiment, the system monitors the indoor environmental parameters of the building in real time, identifies and determines human health characteristics in real time, and accurately identifies suspected infected persons and their close contacts in real time, such that the environmental monitoring means are more comprehensive and the assessment of virus spreading risk in the indoor environment is more accurate. Based on the indoor environmental health effect and the status of suspected infected persons, indoor overall ventilation and purification and local purification treatment are performed to effectively prevent the virus from rapidly spreading in indoor populations and harming people's health.

### Embodiment 2

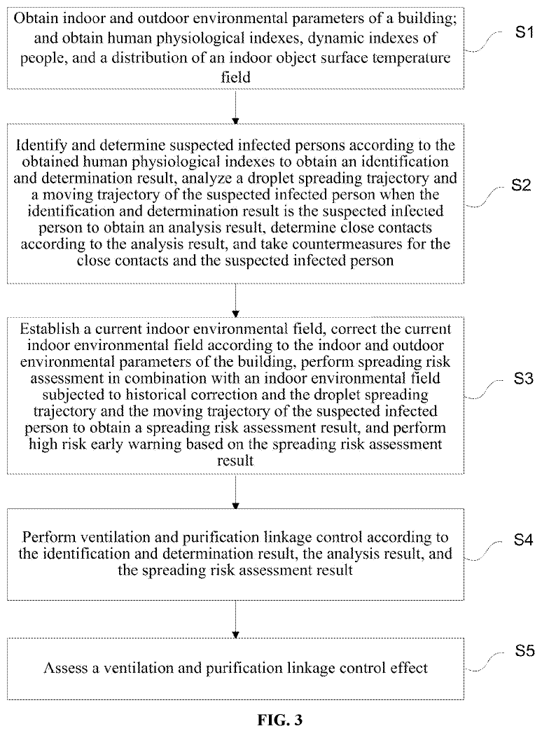

As shown in FIG. 3, a monitoring, tracing, early-warning and control method for virus spreading in a building includes the following steps.

Step S1: Indoor and outdoor environmental parameters of the building are obtained. The environmental parameters include temperature and humidity and a pollutant concentration.

Human physiological indexes, dynamic indexes of people, and a distribution of an indoor object surface temperature field are obtained. The dynamic indexes of people include a droplet spreading trajectory and a moving trajectory of people.

Step S2: Suspected infected persons are identified and determined according to the obtained human physiological indexes to obtain an identification and determination result. A droplet spreading trajectory of the suspected infected person and the moving trajectory of people are analyzed when the identification and determination result is the suspected infected person to obtain an analysis result. Close contacts are determined according to the analysis result. Countermeasures are taken for the close contacts and the suspected infected person.

Step S3: A current indoor environmental field is established. The current indoor environmental field is corrected according to the indoor and outdoor environmental parameters of the building to obtain an indoor environmental field subjected to current correction. Spreading risk assessment is performed in combination with an indoor environmental field subjected to historical correction and the droplet spreading trajectory and the moving trajectory of the suspected infected person to obtain a spreading risk assessment result. High risk early warning is performed based on the spreading risk assessment result. The indoor environmental field subjected to the historical correction is a corrected indoor environmental field before establishment of the current indoor environmental field.

This is implemented specifically in the following sub-steps.

Step S301: A building physical model is established. The distribution of the current indoor environmental parameters is simulated using building environment simulation software in combination with a real-time operating state of building environment control equipment and real-time positioning information of people, so as to establish the current indoor environmental field. The current indoor environmental field includes a temperature and humidity field, a pollutant concentration field, and an air distribution.

Step S302: The established current indoor environmental field is corrected based on the obtained indoor environmental parameters in combination with the distribution of the indoor object surface temperature field, so as to obtain the indoor environmental field subjected to the current correction.

Step S303: An infection probability of indoor virus spreading is calculated according to the indoor environmental field subjected to the current correction, the indoor environmental field subjected to the historical correction, and the droplet spreading trajectory and the moving trajectory of the suspected infected person, so as to assess a spreading risk. A greater infection probability indicates a greater spreading risk.

Step S4: Ventilation and purification linkage control is performed according to the identification and determination result, the analysis result, and the spreading risk assessment result specifically as follows.

Overall control: overall optimization control is performed based on the spreading risk assessment result to realize overall ventilation and purification of the building.

Local control: local ventilation and purification are performed on an area where a concentration of a pollutant in the indoor environmental field subjected to the current correction is higher than a set threshold, and local purification and dilution ventilation are performed on an area where the suspected infected person is located and an area where the close contacts are located to realize local purification treatment of a source.

Step S5: A ventilation and purification linkage control effect is assessed specifically as follows.

An environmental field-time function is established through the indoor environmental field subjected to the current correction and the indoor environmental field subjected to the historical correction, an attenuation rate of the pollutant concentration is calculated, and the ventilation and purification effect is assessed according to the attenuation rate of the pollutant concentration and a current pollutant concentration.

In the present embodiment, according to the method, indoor and outdoor environmental parameters of the building can be monitored in real time. Human physiological indexes can be obtained in real time to determine a human health state, and moving trajectories of people and droplet spreading trajectories are obtained. Therefore, suspected infected persons and their close contacts are timely and accurately identified, and countermeasures are timely taken for the suspected infected persons and the close contacts. In addition, a spreading risk in a current indoor environment can be assessed, such that reliable ventilation and purification measures can be further taken for the indoor environment to minimize the risk of virus spreading and ensure the health and safety of people.

The above description of embodiments is merely provided to help understand the method of the present disclosure and a core idea thereof. It should be noted that several improvements and modifications may be made by those of ordinary skill in the art without departing from the principle of the present disclosure, and these improvements and modifications should also fall within the protection scope of the present disclosure. Various amendments to these embodiments are apparent to those of professional skill in the art, and the general principles defined herein may be implemented in other embodiments without departing from the spirit or scope of the present disclosure. Thus, the present disclosure is not limited to the embodiments shown herein but falls within the widest scope consistent with the principles and novel features disclosed herein.

What is claimed is:

1. A monitoring, tracing, early-warning and control system for virus spreading in a building, comprising an environmental monitoring system, a virus tracing system, an environmental diagnosis and early warning system, and a ventilation and purification linkage control system, wherein the environmental monitoring system comprises a static environmental monitoring system and a dynamic monitoring system;
the static environmental monitoring system is configured to monitor indoor and outdoor environmental parameters of the building, wherein the environmental parameters comprise temperature and humidity and a pollutant concentration;
the dynamic monitoring system is configured to monitor human physiological indexes, dynamic indexes of people, and a distribution of an indoor object surface temperature field, wherein the dynamic indexes of people comprise a droplet spreading trajectory and a moving trajectory of people;
the virus tracing system is configured to identify and determine suspected infected persons according to the obtained human physiological indexes to obtain an identification and determination result, analyze a droplet spreading trajectory of the suspected infected person and the moving trajectory of people when the identification and determination result is the suspected infected person to obtain an analysis result, determine close contacts according to the analysis result, and take countermeasures for the close contacts and the suspected infected person;
the environmental diagnosis and early warning system is configured to establish a current indoor environmental field, correct the current indoor environmental field according to the indoor and outdoor environmental parameters of the building to obtain an indoor environmental field subjected to current correction, perform spreading risk assessment in combination with an indoor environmental field subjected to historical correction and the droplet spreading trajectory and the moving trajectory of the suspected infected person to obtain a spreading risk assessment result, and perform high risk early warning based on the spreading risk assessment result, wherein the indoor environmental field subjected to the historical correction is a corrected indoor environmental field before establishment of the current indoor environmental field; and
the ventilation and purification linkage control system is configured for ventilation and purification linkage control according to the identification and determination result, the analysis result, and the spreading risk assessment result.

2. The monitoring, tracing, early-warning and control system for virus spreading in a building according to claim 1, wherein the static environmental monitoring system comprises a temperature sensor, a humidity sensor, and a pollutant concentration measurement sensor; and the dynamic monitoring system comprises an infrared camera.

3. The monitoring, tracing, early-warning and control system for virus spreading in a building according to claim 1, wherein the environmental diagnosis and early warning system comprises an indoor environmental field establishment module, an indoor environmental field correction module, and a spreading risk assessment module;
the indoor environmental field establishment module is configured to simulate a distribution of the indoor environmental parameters using building environment simulation software, so as to establish the current indoor environmental field;
the indoor environmental field correction module is configured to correct the established current indoor environmental field based on the indoor environmental parameters obtained by the static environmental monitoring system in combination with the distribution of the object surface temperature field, so as to obtain the indoor environmental field subjected to the current correction; and
the spreading risk assessment module is configured to calculate an infection probability of indoor virus spreading according to the indoor environmental field subjected to the current correction, the indoor environmental field subjected to the historical correction, and the droplet spreading trajectory and the moving trajectory of the suspected infected person, so as to assess a spreading risk, wherein a greater infection probability indicates a greater spreading risk.

4. The monitoring, tracing, early-warning and control system for virus spreading in a building according to claim 3, wherein the indoor environmental field establishment module is configured to simulate a distribution of the indoor environmental parameters using building environment simulation software, so as to establish the current indoor environmental field; specifically, the indoor environmental field establishment module is configured to:
establish a building physical model, and simulate the distribution of the current indoor environmental parameters using the building environment simulation software in combination with a real-time operating state of building environment control equipment and real-time positioning information of people, so as to establish the current indoor environmental field, wherein the current indoor environmental field comprises a temperature and humidity field, a pollutant concentration field, and an air distribution.

5. The monitoring, tracing, early-warning and control system for virus spreading in a building according to claim 1, wherein the ventilation and purification linkage control system comprises an overall control system and a local control system;
the overall control system is configured for overall optimization control based on the spreading risk assessment result to realize overall ventilation and purification of the building; and
the local control system is configured for local ventilation and purification of an area where a concentration of a pollutant in the indoor environmental field subjected to the current correction is higher than a set threshold, and local purification and dilution ventilation of an area where the suspected infected person is located and an area where the close contacts are located to realize local purification treatment of a source.

6. The monitoring, tracing, early-warning and control system for virus spreading in a building according to claim 1, further comprising a ventilation and purification effect assessment system configured to assess a ventilation and purification effect of the ventilation and purification linkage control system; specifically, the ventilation and purification effect assessment system is configured to:
establish an environmental field-time function through the indoor environmental field subjected to the current correction and the indoor environmental field subjected to the historical correction, calculate an attenuation rate of the pollutant concentration, and assess the ventilation and purification effect according to the attenuation rate of the pollutant concentration and a current pollutant concentration.

7. A monitoring, tracing, early-warning and control method for virus spreading in a building, comprising the following steps:
step I: obtaining indoor and outdoor environmental parameters of the building, wherein the environmental parameters comprise temperature and humidity and a pollutant concentration; and
obtaining human physiological indexes, dynamic indexes of people, and a distribution of an indoor object surface temperature field, wherein the dynamic indexes of people comprise a droplet spreading trajectory and a moving trajectory of people;
step II: identifying and determining suspected infected persons according to the obtained human physiological indexes to obtain an identification and determination result, analyzing a droplet spreading trajectory of the suspected infected person and the moving trajectory of people when the identification and determination result is the suspected infected person to obtain an analysis result, determining close contacts according to the analysis result, and taking countermeasures for the close contacts and the suspected infected person;
step III: establishing a current indoor environmental field, correcting the current indoor environmental field according to the indoor and outdoor environmental parameters of the building to obtain an indoor environmental field subjected to current correction, performing spreading risk assessment in combination with an indoor environmental field subjected to historical correction and the droplet spreading trajectory and the moving trajectory of the suspected infected person to obtain a spreading risk assessment result, and performing high risk early warning based on the spreading risk assessment result, wherein the indoor environmental field subjected to the historical correction is a corrected indoor environmental field before establishment of the current indoor environmental field;
step IV: performing ventilation and purification linkage control according to the identification and determination result, the analysis result, and the spreading risk assessment result; and
step V: assessing a ventilation and purification linkage control effect.

8. The monitoring, tracing, early-warning and control method for virus spreading in a building according to claim 7, wherein a process of correcting the current indoor environmental field according to the indoor and outdoor environmental parameters of the building and performing spreading risk assessment in combination with an indoor environmental field subjected to historical correction and the droplet spreading trajectory and the moving trajectory of the suspected infected person to obtain a spreading risk assessment result specifically comprises:
establishing a building physical model, and simulating the distribution of the current indoor environmental parameters using building environment simulation software in combination with a real-time operating state of building environment control equipment and real-time positioning information of people, so as to establish the current indoor environmental field, wherein the current indoor environmental field comprises a temperature and humidity field, a pollutant concentration field, and an air distribution;
correcting the established current indoor environmental field based on the obtained indoor environmental parameters in combination with the distribution of the indoor object surface temperature field, so as to obtain the indoor environmental field subjected to the current correction; and
calculating an infection probability of indoor virus spreading according to the indoor environmental field subjected to the current correction, the indoor environmental field subjected to the historical correction, and the droplet spreading trajectory and the moving trajectory of the suspected infected person, so as to assess a spreading risk, wherein a greater infection probability indicates a greater spreading risk.

9. The monitoring, tracing, early-warning and control method for virus spreading in a building according to claim 7, wherein a process of performing ventilation and purification linkage control according to the identification and determination result, the analysis result, and the spreading risk assessment result specifically comprises:
overall control: performing overall optimization control based on the spreading risk assessment result to realize overall ventilation and purification of the building; and
local control: performing local ventilation and purification on an area where a concentration of a pollutant in the indoor environmental field subjected to the current correction is higher than a set threshold and local purification and dilution ventilation on an area where the suspected infected person is located and an area where the close contacts are located to realize local purification treatment of a source.

10. The monitoring, tracing, early-warning and control method for virus spreading in a building according to claim 7, wherein a process of assessing a ventilation and purification linkage control effect specifically comprises:
establishing an environmental field-time function through the indoor environmental field subjected to the current correction and the indoor environmental field subjected to the historical correction, calculating an attenuation rate of the pollutant concentration, and assessing the ventilation and purification effect according to the attenuation rate of the pollutant concentration and a current pollutant concentration.
